(19) United States
(12) Patent Application Publication
(10) Pub. No.: US 2010/0004289 A1
(43) Pub. Date: Jan. 7, 2010

(54) SUBSTITUTED BUTYROPHENONE DERIVATIVES

(75) Inventor: Philip Seeman, Toronto (CA)

Correspondence Address: BERESKIN AND PARR LLP/S.E.N.C.R.L., s.r.l., 40 KING STREET WEST, BOX 401, TORONTO, ON, M5H 3Y2, CA

(73) Assignee: CLERA INC., Toronto, ON (CA)

(21) Appl. No.: 11/912,999

(22) PCT Filed: May 1, 2006
(86) PCT No.: PCT/CA2006/000672

§ 371 Date: Sep. 25, 2009

Related U.S. Application Data
(60) Provisional application No. 60/675,881, filed on Apr. 29, 2005.

(51) Int. Cl. A61K 31/445; C07D 211/44; A61P 25/00

(52) U.S. Cl. 514/327; 546/217

(57) ABSTRACT

The present invention relates to a central nervous system-acting substituted butyrophenones. These compounds are useful in antipsychotic medications for psychosis, including schizophrenia, but especially for L-DOPA-induced psychosis, while having low or no risk of eliciting extrapyramidal side effects, hyperprolactinemia or tardive dyskinesia.

### FIELD OF THE INVENTION

The present invention relates to a central nervous system-acting substituted butyrophenone derivatives. These compounds are useful as antipsychotic medication for psychosis, including schizophrenia, but especially for L-DOPA-induced psychosis in Parkinson's diseased patients, while having low or no risk of eliciting extrapyramidal side effects, hyperprolactinemia or tardive dyskinesia.

### BACKGROUND OF THE INVENTION

Psychosis occurs in many mental illnesses, including schizophrenia, Huntington's disease, Alzheimer's disease and in individuals who take L-DOPA for Parkinson's disease. Although the biological causes for these forms of psychosis are not known, it is known that antipsychotic drugs which block dopamine D2 receptors can block or reduce the psychotic symptoms in all of these forms of psychosis. The dopamine-blocking action of the antipsychotics suggests that psychosis is usually associated with over-active dopamine neurotransmission. As stated by Su et al. (Arch. Gen. Psychiat. 54: 972-973, 1997), “ . . . no drug has yet been identified with antipsychotic action without a significant affinity for the D2 receptor.” In the special case of Parkinson's disease, such patients take high doses of oral L-DOPA to alleviate their immobilities, thus readily eliciting psychosis. Although all antipsychotic drugs can block L-DOPA-induced psychosis, these drugs intensify the Parkinsonian signs of akinesia and rigidity. While clozapine and quetiapine are exceptions and do not worsen the Parkinsonian signs, clozapine can cause leucopenia, and quetiapine can cause excessive sedation. Therefore, in order to treat L-DOPA psychosis, there is a need for antipsychotics which have the advantages of clozapine and quetiapine but which do not have the disadvantages.

Traditional antipsychotics (such as chlorpromazine, haloperidol, and trifluperazine) can induce unwanted clinical side effects such as Parkinsonism, elevated serum prolactin and breast swelling, drowsiness, and late-appearing tardive dyskinesia. Most of the side effects are associated with the basic mechanism of action of antipsychotic compounds, which is to block dopamine D2 receptors.

“Atypical” antipsychotic drugs do not elicit these side effects, or elicit them with much less intensity, or elicit them only at high doses. As indicated above, all antipsychotic compounds operate primarily by attaching to, and blocking, dopamine D2 receptors in the brain. It is believed that atypical antipsychotics may clinically help patients by transiently occupying D2 receptors and then rapidly dissociating to allow normal dopamine neurotransmission. According to this theory, drugs that bind more loosely than dopamine to the dopamine D2 receptor, and, therefore, have dissociation constants higher than that of dopamine, are likely to exhibit fewer side effects than traditional antipsychotics. The mechanism of action of traditional and atypical antipsychotic drugs is described in P. Seeman, Can. J. Psychiat. Vol. 47(1): 27-38, 2002.

There is a need for new atypical antipsychotics which are clinically effective in alleviating psychotic symptoms but which do not have side effects and, in the special case of L-DOPA-induced psychosis in Parkinson's-diseased patients, do not worsen the Parkinsonian signs and symptoms.

### SUMMARY OF THE INVENTION

This application relates to certain substituted butyrophenone derivatives useful as atypical antipsychotics for treatment of psychosis and related mental disorders, especially for L-DOPA-induced psychosis. When used in antipsychotic medications, these compounds do not exhibit deleterious or unwanted side effects, such as extrapyramidal signs, hyperprolactinemia and tardive dyskinesia. The compounds may be present in the form of a free base or as a pharmaceutically acceptable acid addition salts and may be combined with a pharmaceutically acceptable carrier.

Accordingly, one aspect of the present invention includes a compound selected from a compound of formula (I):

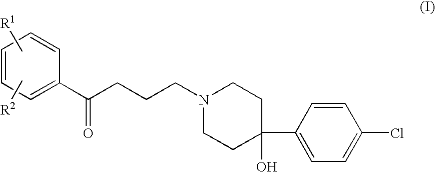

wherein
R¹ is selected from the group consisting of OC₁₋₆alkyl, fluoro-substituted OC₁₋₄alkyl and OH; and
R² is selected from the group consisting of H and fluoro;
and pharmaceutically acceptable acid addition salts and solvates thereof,
with the proviso that when R¹ is OCH₃, R¹ is attached at the 3-position of the phenyl ring.

The present invention also includes a pharmaceutical composition comprising a compound of the invention and pharmaceutically acceptable carriers or diluents.

Also included within the scope of the present invention is a method of treating psychosis comprising administering an effective amount of a compound of the invention to a subject in need thereof. Further the invention includes a use of a compound of the invention to treat psychosis, as well as a use of a compound of the invention to prepare a medicament to treat psychosis.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION

This application relates to new atypical antipsychotic compounds, namely certain substituted butyrophenone derivatives and pharmaceutically acceptable salts and solvates thereof.

Accordingly, in one of its aspect, the present invention includes a compound selected from a compound of Formula (I):

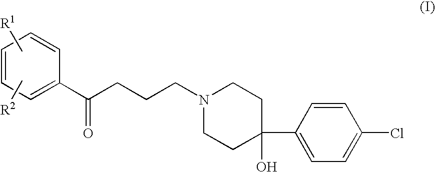

wherein
R¹ is selected from the group consisting of OC₁₋₆alkyl, halo-substituted OC₁₋₄alkyl and OH; and
R² is selected from the group consisting of H and fluoro;
and pharmaceutically acceptable acid addition salts and solvates thereof,
with the proviso that when R¹ is OCH₃, R¹ is attached at the 3-position of the phenyl ring.

The compounds of Formula I include those in which R¹ is selected from the group consisting of OC₁₋₆alkyl, fluoro-substituted OC₁₋₆alkyl and OH. In embodiments of the invention R¹ is selected from the group consisting of OC₁₋₄alkyl, fluoro-substituted OC₁₋₄alkyl and OH. In further embodiments of the invention, R¹ is selected from the group consisting of OCH₃, OCF₃ and OH. In still further embodiments of the invention, R¹ is OCH₃.

The compounds of Formula I include those in which R² is H or F. In embodiments of the invention R² is H. In further embodiments of the invention, R² is F attached at the 4-position of the phenyl ring.

In an embodiment of the invention, the compound of Formula I has the following structure:

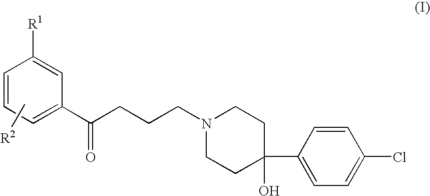

wherein
R¹ is selected from the group consisting of OC₁₋₆alkyl, fluoro-substituted OC₁₋₄alkyl and OH; and
R² is selected from the group consisting of H and fluoro;
and pharmaceutically acceptable acid addition salts and solvates thereof.

In another embodiment of the invention, the compound of Formula I has the following structure:

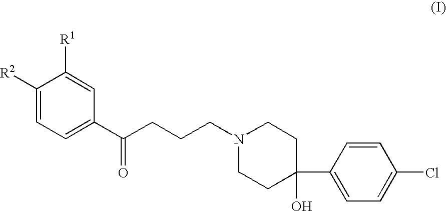

wherein
R¹ is selected from the group consisting of OC₁₋₆alkyl, fluoro-substituted OC₁₋₆alkyl and OH; and
R² is selected from the group consisting of H and fluoro;
and pharmaceutically acceptable acid addition salts and solvates thereof.

In an embodiment of the invention, the compound of Formula I is selected from:
4-[4-(4-chlorophenyl)-4-hydroxypiperidin-1-yl]-1-(4-fluoro-3-methoxyphenyl)butan-1-one;
4-[4-(4-chlorophenyl)-4-hydroxypiperidin-1-yl]-1-(3-methoxyphenyl)butan-1-one;
4-[4-(4-chlorophenyl)-4-hydroxypiperidin-1-yl]-1-(3-trifluoromethoxyphenyl)butan-1-one;
4-[4-(4-chlorophenyl)-4-hydroxypiperidin-1-yl]-1-(3-ethoxyphenyl)butan-1-one;
4-[4-(4-chlorophenyl)-4-hydroxypiperidin-1-yl]-1-(4-fluoro-3-ethoxyphenyl)butan-1-one; and
4-[4-(4-chlorophenyl)-4-hydroxypiperidin-1-yl]-1-(4-fluoro-3-trifluoromethoxyphenyl)butan-1-one, and
   pharmaceutically acceptable acid addition salts and solvates thereof.

In a further embodiment of the invention, the compound of Formula I is selected from:
4-[4-(4-chlorophenyl)-4-hydroxypiperidin-1-yl]-1-(3-methoxyphenyl)butan-1-one;
4-[4-(4-chlorophenyl)-4-hydroxypiperidin-1-yl]-1-(3-trifluoromethoxyphenyl)butan-1-one; and
4-[4-(4-chlorophenyl)-4-hydroxypiperidin-1-yl]-1-(3-ethoxyphenyl)butan-1-one, and
   pharmaceutically acceptable acid addition salts and solvates thereof.

The term “C₁₋ₙalkyl” as used herein means straight and/or branched chain, saturated alkyl radicals containing from one to n carbon atoms and includes (depending on the identity of n) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like.

The term “fluoro-substituted C₁₋ₙalkyl” as used herein means a C₁₋ₙalkyl group in which one or more of the hydrogen atoms has been replaced by F, and includes trifluoromethyl, trifluoroethyl, pentafluoroethyl and the like.

The term “compound(s) of the invention” as used herein means compound(s) of Formula I and/or pharmaceutically acceptable salts and/or solvates thereof.

It is to be clear that the present invention includes pharmaceutically acceptable salts and solvates of the compounds of the Formula I and mixtures comprising two or more of a compound of Formula I, pharmaceutically acceptable salts of a compound of Formula I, and pharmaceutically acceptable solvates of a compound of Formula 1.

The term “pharmaceutically acceptable” means compatible with the treatment of animals, in particular, humans.

The term “pharmaceutically acceptable acid addition salt” as used herein means any non-toxic organic or inorganic salt of any base compound of the invention, or any of its intermediates, which are suitable for or compatible with the treatment of animals, in particular humans. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of the compounds of the invention are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of the compounds of the invention, for laboratory use, or for subsequent conversion to a pharmaceutically acceptable acid addition salt. In an embodiment of the invention, the pharmaceutically acceptable acid addition salt is a hydrochloride salt. The formation of a desired compound salt is achieved using standard techniques. For example, the neutral compound is treated with an acid in a suitable solvent and the formed salt is isolated by filtration, extraction or any other suitable method.

The term “solvate” as used herein means a compound of the invention wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates of the compounds of the invention will vary depending on the compound and the solvate. In general, solvates are formed by dissolving the compound in the appropriate solvent and isolating the solvate by cooling or using an antisolvent. The solvate is typically dried or azeotroped under ambient conditions.

The compounds of Formula I can be prepared using methods known in the art, for example as shown in Scheme 1 and described in the specific examples herein below.

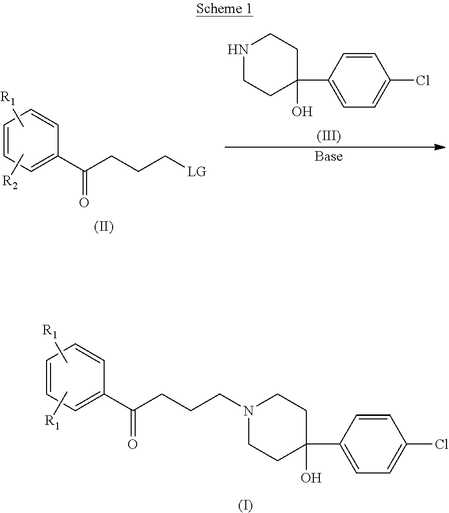

Accordingly, compounds of Formula II, wherein R¹ and R² are as defined in Formula I and LG is a suitable leaving group, such as halo, for example iodo, may be reacted with a compound of Formula III in the presence of a suitable base, under standard nucleophilic substitution reaction conditions, to provide compounds of Formula I.

Compounds of Formula II may be prepared, for example, as shown in Scheme 2, and as described in the specific examples hereinbelow.

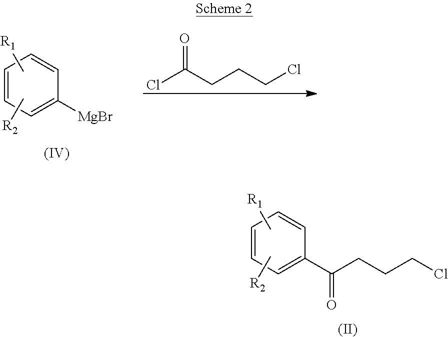

Accordingly, Grignard reagents of Formula IV, wherein R¹ and R² are as defined in Formula I, may be reacted with 4-chlorobutyrylchloride under standard Grignard reaction conditions to provide compounds of Formula II, wherein R¹ and R² are as defined in Formula I and LG is chloro. Compounds of Formula II, wherein LG is chloro may be converted to other compounds of Formula II with alternate LG moieties using standard chemistries.

Compounds of Formula III, IV and 4-chlorobutyrylchloride are either commercially available or may be prepared using methods well known in the art.

The present invention includes radiolabeled forms of the compounds of the invention, for example, compounds of the invention labeled by incorporation within the structure ³H, ¹¹C or ¹⁴C or a radioactive halogen such as ¹²⁵I and ¹⁸F. A radiolabeled compound of the invention may be prepared using standard methods known in the art. For example, tritium may be incorporated into a compound of the invention using standard techniques, for example by hydrogenation of a suitable precursor to a compound of the invention using tritium gas and a catalyst. Alternatively, a compound of the invention containing radioactive iodo may be prepared from the corresponding trialkyltin (suitably trimethyltin) derivative using standard iodination conditions, such as [¹²⁵I] sodium iodide in the presence of chloramine-T in a suitable solvent, such as dimethylformamide. The trialkyltin compound may be prepared from the corresponding non-radioactive halo, suitably iodo, compound using standard palladium-catalyzed stannylation conditions, for example hexamethylditin in the presence of tetrakis(triphenylphosphine) palladium (0) in an inert solvent, such as dioxane, and at elevated temperatures, suitably 50-100° C. Further, a compound of the invention containing a radioactive fluorine may be prepared, for example, by reaction of K[¹⁸F]/K222 with a suitable precursor compound, such as a compound of Formula I comprising a suitable leaving group, for example a tosyl group, that may be displaced with the ¹⁸F anion.

Parkinson's diseased patients have only between 0.3% and 2% of normal levels of dopamine remaining in their caudate nucleus, and even lower concentrations of between 0.1% to 1% in the putamen. Such patients, therefore, need to take very high doses of L-DOPA to replenish their brain dopamine in order to alleviate their akinesia and rigidity. These high doses usually elicit psychotic symptoms which are very troublesome to the patient and which need to be treated. For this type of situation, it is desirable to administer an antipsychotic which is extremely loosely bound, and which, therefore, has a high K value, for example, of the order of 30 to 160 nM. Compounds with such a high K block dopamine D2 receptors very briefly, interrupting and preventing hallucinations, but not worsening the Parkinsonian rigidity and akinesia.

It is well known in neurology that L-DOPA psychosis in a Parkinson's diseased patient is best treated with a dose of clozapine which is about 5% or 10% the dose normally used for psychosis in schizophrenia. The “fast-off-D2” hypothesis readily and quantitatively predicts this, as follows. The antipsychotic dose needed to occupy D2 receptors is proportional to K×[1+D/Dhigh], where K is the dissociation constant of the antipsychotic, D is the concentration of dopamine in the synaptic space during the momentary nerve impulse (˜200 nM), and where Dhigh is the dissociation constant of dopamine at the high-affinity state of D2 (˜1.75 nM). In Parkinson's disease, where 95% to 99% of the dopamine content is absent, the value for D would be 10 nM. Accordingly, the antipsychotic dose for L-DOPA psychosis will be lower than that for schizophrenia psychosis by a factor of {1+D/Dhigh}ₙₒᵣₘₐₗ/{1+D/Dhigh}_{Parkinson} or {1+200/1.75}/{1+10/1.75} or 20-fold. Thus, while a daily dose of 500 mg clozapine might be suitable for treating schizophrenia psychosis, one-twentieth of this dose, namely 25 mg (or less) would be more than adequate to treat L-DOPA psychosis. This calculation best holds for competition between endogenous dopamine and a loosely bound antipsychotic. A tightly bound antipsychotic such as haloperidol would not readily permit endogenous dopamine to replace it competitively.

Certain compounds of the invention have a K value of the order of 140±8 nM, putting them in the optimum range for treating L-DOPA psychosis. This value is optimum because it indicates that the molecule is approximately 80 times more loosely bound to the rat brain or human brain dopamine D2 receptor than is dopamine itself, where dopamine has an affinity of 1.75 nM for its own D2 receptor. A compound having such characteristics has been shown to avoid eliciting catalepsy or elevating prolactin in rats, and would readily block conditioned avoidance behaviour.

Accordingly, the present invention further includes a method of treating psychosis comprising administering an effective amount of a compound of the invention to a subject in need thereof. The invention also includes a use of a compound of the invention to treat psychosis and a use of a compound of the invention to prepare a medicament to treat psychosis.

The term an “effective amount” or a “sufficient amount” of an agent as used herein is that amount sufficient to effect beneficial or desired results, including clinical results, and, as such, an “effective amount” depends upon the context in which it is being applied. For example, in the context of administering an agent that treats psychosis, an effective amount of an agent is, for example, an amount sufficient to achieve such a treatment as compared to the response obtained without administration of the agent.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder.

The term “subject” as used herein includes all members of the animal kingdom including human. The subject is suitably human.

The term “psychosis” as used herein refers to any psychiatric disorder that is marked by schizophrenia-like symptoms including, for example, delusions, hallucinations, incoherence and distorted perceptions of reality. Psychosis occurs in many mental illnesses, including schizophrenia, Huntington's disease, Alzheimer's disease and in individuals who take L-DOPA for Parkinson's disease. In an embodiment of the invention, the psychosis is L-DOPA-induced psychosis.

The compounds of the invention are suitably formulated into pharmaceutical compositions for administration to human subjects in a biologically compatible form suitable for administration in vivo. Accordingly, in another aspect, the present invention includes a pharmaceutical composition comprising a compound of the invention and a pharmaceutically acceptable carrier or diluent.

The compositions containing the compounds of the invention can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (2003-20th edition) and in The United States Pharmacopeia: The National Formulary (USP 24 NF19) published in 1999. On this basis, the compositions include, albeit not exclusively, solutions of the substances in association with one or more pharmaceutically acceptable vehicles or diluents, and contained in buffered solutions with a suitable pH and iso-osmotic with the physiological fluids.

In accordance with the methods of the invention, the described compounds, salts or solvates thereof may be administered to a patient in a variety of forms depending on the selected route of administration, as will be understood by those skilled in the art. The compositions of the invention may be administered, for example, by oral, parenteral, buccal, sublingual, nasal, rectal, patch, pump or transdermal (topical) administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound of the invention may be incorporated with excipient and used in the form of ingestible tablets, buccal tablets, troches, capsules, elixirs, suspensions, syrups, wafers, and the like.

A compound of the invention may also be administered parenterally. Solutions of a compound of the invention can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists. Ampoules are convenient unit dosages.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Compositions for topical administration may include, for example, propylene glycol, isopropyl alcohol, mineral oil and glycerin. Preparations suitable for topical administration include liquid or semi-liquid preparations such as liniments, lotions, applicants, oil-in-water or water-in-oil emulsions such as creams, ointments or pastes; or solutions or suspensions such as drops. In addition to the aforementioned ingredients, the topical preparations may include one or more additional ingredients such as diluents, buffers, flavouring agents, binders, surface active agents, thickeners, lubricants, preservatives, e.g. methyl hydroxybenzoate (including anti-oxidants), emulsifying agents and the like.

Sustained or direct release compositions can be formulated, e.g. liposomes or those wherein the active compound is protected with differentially degradable coatings, such as by microencapsulation, multiple coatings, etc. It is also possible to freeze-dry the compounds of the invention and use the lypolizates obtained, for example, for the preparation of products for injection.

The compounds of the invention may be administered to a subject alone or in combination with pharmaceutically acceptable carriers, as noted above, and/or with other pharmaceutically active agents for the treatment of psychosis, the proportion of which is determined by the solubility and chemical nature of the compounds, chosen route of administration and standard pharmaceutical practice.

The dosage of the compounds and/or compositions of the invention can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. Oral preparations may be formulated, preferably as tablets, capsules, or drops, containing from 5-300 milligrams of a compound of the invention, per dosage unit. The compounds of the invention may be administered initially in a suitable dosage that may be adjusted as required, depending on the clinical response.

In addition to the above-mentioned therapeutic uses, the compounds of the invention are also useful in diagnostic assays, screening assays and as research tools.

In diagnostic assays the compounds of the invention may be useful in identifying or detecting the dopamine D₂ receptor. In such an embodiment, the compounds of the invention may be radiolabelled (as hereinbefore described) and contacted with a population of cells. The presence of the radiolabel on the cells may indicate the presence of the dopamine D₂ receptor.

In screening assays, the compounds of the invention may be used to identify other compounds that bind to the dopamine D₂ receptor. As research tools, the compounds of the invention may be used in receptor binding assays and assays to study the localization of the dopamine D₂ receptor. In such assays, the compounds may also be radiolabelled.

While the following Examples illustrate the invention in further detail, it will be appreciated that the invention is not limited to the specific Examples.

### EXAMPLES

All reactions were performed under an argon atmosphere. All solvents and reagents were obtained from commercial sources and used without any further purification. Chromatographic purification was performed using 60 Å (230-400 mesh) silica gel. NMR spectra were recorded on 300 MHz spectrometer.

### Example 1(a)

### 4-Chloro-1-(3-methoxyphenyl)-butan-1-one

A solution of 4-chlorobutyrylchloride (1.59 mL, 14.194 mmol) in dry THF (20 mL) was treated with 3-methoxy phenylmagnesium bromide (14.19 mL, 14.194 mmol, 1M solution in THF) at −20° C. over a period of 30 min. The reaction was quenched with saturated NH₄Cl solution (25 mL) after stirring for additional 10 min. The reaction mixture was brought to room temperature and diluted with water. The compound was extracted into ethyl acetate (2×25 mL), washed with water (20 mL), brine (15 mL) and dried (Na₂SO₄). The ethyl acetate layer was evaporated and the crude was purified by column chromatography (EtOAc:Hexanes, 8:92) to obtain the title compound (1.1 g, 36%) as a syrup. ¹H NMR (CDCl₃) δ 2.18-2.27 (m, 2H), 3.17 (t, 2H, J=6.9 Hz), 3.68 (t, 2H, J=6.3 Hz), 3.86 (s, 3H), 7.12 (dd, 1H, J=3.0, 7.6 Hz), 7.38 (t, 1H, J=8.1 Hz), 7.49 (t, 1H, J=1.8 Hz), 7.56 (d, 1H, J=7.5 Hz); MS-ESI (m/z, %) 213 (M⁺, 100), 177 (54).

In a like manner, the following additional compounds may be prepared:
(b) 4-Chloro-1-(4-fluoro-3-methoxyphenyl)-butan-1-one, from 4-fluoro-3-methoxy phenylmagnesium bromide;
(c) 4-Chloro-1-(3-trifluoromethoxyphenyl)-butan-1-one, from 3-trifluoromethoxy phenylmagnesium bromide;
(d) 4-Chloro-(3-ethoxyphenyl)-butan-1-one, from 3-ethoxy phenylmagnesium bromide;
(e) 4-Chloro-(4-fluoro-3-ethoxyphenyl)butan-1-one, from 4-fluoro-3-ethoxy phenylmagnesium bromide; and
(f) 4-chloro-(4-fluoro-3-trifluoromethoxyphenyl)butan-1-one, from 4-fluoro-3-trifluoromethoxy phenylmagnesium bromide.

### Example 2(a)

### 4-[4-(4-chlorophenyl)-4-hydroxypiperidin-1-yl]-1-(3-methoxyphenyl)butan-1-one

A solution of 4-chloro-1-(3-methoxyphenyl)-butan-1-one (Example 1(a), 0.1 g, 0.470 mmol) in acetone (5 mL) was treated with NaI (0.35 g, 2.351 mmol) at room temperature and resulting mixture was refluxed for overnight (14 h). The reaction mixture was brought to room temperature and the solvent was evaporated under vacuum. The reaction mixture was diluted with water (25 mL) and the product extracted into ether (2×25 mL). The combined ether layers were washed with water (25 mL), brine (20 mL) and dried (Na₂SO₄). The solvent was evaporated under reduced pressure to obtain the crude iodo compound.

A solution of the above crude compound in acetone (5 mL), was treated with 4-(4-chlorophenyl)piperidin-4-ol (0.1 g, 0.470 mmol), K₂CO₃ (0.13 g, 0.940 mmol) and the resulting mixture was refluxed for 48 h. The reaction mixture was worked up and purified as described above in part 1, to obtain the title compound (0.14 g, 77%) as a solid. mp 127-129° C.; ¹H NMR (CD₃OD) δ 1.64-1.68 (m, 2H), 1.92-2.02 (m, 4H), 2.50-2.57 (m, 4H), 2.80-2.83 (m, 2H), 3.05 (t, 2H, J=6.6 Hz), 3.85 (s, 3H), 7.17 (dd, 1H, J=3.0, 8.1 Hz), 7.29-7.32 (m, 2H), 7.40-7.45 (m, 3H), 7.52-7.53 (m, 1H), 7.61 (d, 1H, J=7.8 Hz); MS-ESI (m/z, %) 388 (M⁺, 100).

In a like manner, the following additional compound was prepared:
(b) 4-[4-phenyl-4-hydroxypiperidin-1-yl]-1-(3-methoxyphenyl)butan-1-one, from Example 1(a) and 4-phenylpiperidin-4-ol (comparative example);
   and the following additional compounds may be prepared:
(c) 4-[4-(4-chlorophenyl)-4-hydroxypiperidin-1-yl]-1-(4-fluoro-3-methoxyphenyl)butan-1-one, from Example 1(b);
(d) 4-[4-(4-chlorophenyl)+hydroxypiperidin-1-yl]-1-(3-trifluoromethoxyphenyl)butan-1-one, from Example 1(c);
(e) 4-[4-(4-chlorophenyl)-4-hydroxypiperidin-1-yl]-1-(3-ethoxyphenyl)butan-1-one, from Example 1(d);
(f) 4-[4-(4-chlorophenyl)-4-hydroxypiperidin-1-yl]-1-(4-fluoro-3-ethoxyphenyl)butan-1-one, from Example 1(e);
(g) 4-[4-(4-chlorophenyl)-4-hydroxypiperidin-1-yl]-1-(4-fluoro-3-trifluoromethoxyphenyl)butan-1-one, from Example 1(f).

### Example 3

### In Vitro Tests

A compound of the present invention was compared to known antipsychotic compounds in in vitro tests.

### (a) Tissues

Rat brains were purchased from Pel-Freez (Rogers, Ark.) and stored at −70° C. The rat brain striatum was used to measure the binding of drugs to dopamine D1 and D2 receptors, while the rat frontal cerebral cortex was used for serotonin-1 receptors, serotonin-2A receptors, alpha-2A-adrenoceptors and beta-2-adrenoceptors. Before each experiment, the striatum or the frontal cerebral cortex (free of myelin) was dissected from the partly thawed rat brain on a glass plate on a bed of dry ice. The dissected tissue was suspended in buffer (50 mM Tris-HCl, pH 7.4 at 20° C., 1 mM EDTA, 5 mM KCl, 120 mM NaCl, 1.5 mM CaCl₂, 4 mM MgCl₂) at 4 mg original wet weight per ml suspension. The suspension was homogenized for 5 s with a Polytron (PT-10 probe, Brinkmann Instruments, Inc, Westbury, N.Y.; setting 5) without any subsequent washing, centrifugation or preincubation, because such procedures result in a loss of 23-37% of receptors (J. Neurochem. 43: 221-235, 1984).

### (b) Cloned Receptors in Tissue Culture Cells

Human dopamine D1 receptors (in Sf9 cells or COS cells), human dopamine D2Long receptors (in Sf9 cells or CHO cells), human alpha-adrenoceptor-2A receptors, and human muscarinic M1 receptors, all expressed in Sf9 cells, were purchased from Research Biochemicals International (Natick, Mass.). The frozen membranes containing the receptors were directly suspended at approximately 100 μg protein/ml and the cell suspension was homogenized for 5 s (Polytron, setting 5) without any further washing.

### (c) [³H]Ligands

[N-methyl-3H]SCH23390 (70-87 Ci/mmol), [³H]raclopride (70-80 Ci/mmol), [³H]QNB or L-[N-methyl-3H]quinuclidinyl benzilate methyl chloride (84 Ci/mmol), [³H]8-OH-DPAT or [³H]-8-hydroxy-dipropylaminotetralin (163 Ci/mmol), [³H]prazosin (80 Ci/mmol), [³H]yohimbine (71 Ci/mmol), [³H]dihydroalprenolol (106 Ci/mmol), [ethylene-3H]ketanserin (60-90 Ci/mmol) and [³⁵S]GTP-gamma-S {or [³⁵S]guanosine-5′-(gamma-thio)triphosphate} (1,250 Ci/mmol) were purchased from New England Nuclear Life Science Products (through Mandel, Guelph, Ontario, Canada).

### (d) Competitive Binding Assays

The competition between a compound and a [³H]ligand for binding at the various receptors was done as follows. Each incubation tube (12×75 mm glass) received, in the following order, 0.5 ml buffer (50 mM Tris-HCl, pH 7.4 at 20° C., 1 mM EDTA, 5 mM KCl, 120 mM NaCl, 1.5 mM CaCl₂, 4 mM MgCl₂) containing a range of drug concentrations (final concentrations of 0.01 nM to 1,000 nM) or an excess of a second drug in order to define nonspecific binding, followed by the addition of 0.25 ml [³H]ligand, and 0.25 ml of homogenized tissue suspension. The tubes, containing a total volume of 1 ml, were incubated for 2 h at room temperature (20° C.), after which the incubates were filtered, using a 12-well cell harvester (Titertek, Skatron, Lier, Norway) and buffer-presoaked glass fiber filter mats (No. 11734, Skatron, Sterling, Va.). After filtering the incubate, the filter mat was rinsed with buffer for 15 s (7.5 ml buffer). The filters were pushed out and placed in scintillation minivials (Packard Instruments, Chicago, Ill.). The minivials received 4 ml each of scintillant (CytoScint, ICN, CA), and were monitored 6 h later for tritium in a Beckman Coulter LS5000TA scintillation spectrometer at 55% efficiency.

The competitive potencies of the compounds at the cloned dopamine D1 receptors were measured using a final concentration of 1.25 nM [³H]SCH23390 (Kd was 0.5 nM) and using 1 μM (+)-butaclamol to define nonspecific binding. Drug competition at the cloned dopamine D2 receptors (either D2short or D2 long) were measured using 2 nM [³H]raclopride (Kd was 1.9 nM) and using 10 μM S-sulpiride to define nonspecific binding. Competition at the muscarinic receptors was done using either the cloned M1 receptors or the rat frontal cortex, 0.6 nM [³H]QNB, and using 200 nM atropine to define nonspecific binding. Competition at the cloned serotonin-1A receptors was done with 1.4 nM [³H]8-OH-DPAT (Kd was 1.5 nM) and using 100 μM serotonin to define nonspecific binding. Competition at the serotonin-2A receptors was done using either rat frontal cerebral cortex tissue or cloned serotonin-2A receptors, 1 nM [³H]ketanserin, and using 10 μM serotonin to define nonspecific binding; the cortex and the cloned receptors gave very similar results. Competition at alpha-1-adrenoceptors was done using rat cerebral cortex tissue, 1.5 nM [³H]prazosin, and using 10 μM adrenaline to define nonspecific binding. Competition at alpha-2A-adrenoceptors was done using human cloned rat receptors (in Sf9 cells), 2.1 nM [³H]yohimbine and using 100 μM adrenaline to define nonspecific binding. Competition at beta-adrenoceptors was done using rat cerebral cortex tissue, 0.5 nM [³H]dihydroalprenolol, and using 200 nM propranolol to define nonspecific binding. Competition at sigma receptors was done using rat striatal tissue and 4 nM [³H]haloperidol (Kd=1 nM haloperidol) and 1 μM (+)pentazocine as baseline (using this latter test, haloperidol itself had a Ki of ˜3 nM on the sigma receptor). The compound dissociation constant, K, was calculated as usual as C50%/[1+C*/Kd], where C50% was the drug concentration which inhibited ligand binding by 50%, where C* was the ligand concentration, and where Kd was the dissociation constant of the ligand, as obtained from a separate experiment using a range of ligand concentrations.

### (e) Method for Measuring D2 Occupancy In Vivo:

Sprague-Dawley rats (each 250 g) received an oral dose of test compound of 20 mg/kg by gavage, using 2 ml per gavage feeding. The test compound solution was prepared as follows: 20 mg of test compound was added to 8 ml saline (0.9% NaCl), followed by the addition of a few drops of 2% lactic acid and back titration of the suspension to pH 5 using 0.1 N NaOH. A fine particulate suspension was used for gavage. Each rat received 2 ml of the suspension over a period of 5 minutes. After 30 min, after 1 h, after 2 h and after 3 h, each rat received an injection of 7.5 μCi (300 μL or 0.3 ml diluted from the stock of [³H]raclopride (prepared by PerkinElmer Life Science, Boston, Mass.) into the warmed tail vein. 1 hour after each injection of [³H]raclopride, the rat head was removed by guillotine, and the brain and striata and cerebellum removed. The cerebellum and striata were chopped into several large pieces. The tissues remained overnight in scintillation fluid, allowing extraction of the [³H]raclopride. Samples were counted the next day in a Beckman scintillation spectrometer. The D2 occupancy in the striatum was calculated. Two control rats without any test compound had a binding potential of 9.23.

### (f) Method for Measuring Catalepsy:

The front paws of the animal were placed on a horizontal bar. Control animals quickly moved off the bar. Cataleptic animals held on to the bar for up to 15 seconds or more.

### (g) In Vitro Test Results

The dissociation constants of the compound of Example 2(a) were 140±8 nM at the cloned dopamine human D2Long receptor, 570 nM at the muscarinic cholinergic receptor (rat cortex tissue), 12,000 nM at the serotonin-1A receptor (rat frontal cortex tissue), 11,000 nM at the dopamine D1 receptor (rat striatal tissue), 1,067 nM at the alpha-1-adrenoceptor (rat cortex), 9,500 nM at the alpha-2-adrenoceptor (cloned in Sf9 cells), 1,141 nM at the histamine H1 receptor (rat cortex), 2,600 nM at the HERG channel receptor (using 6 nM [3H]dofetilide on rat striatal tissue; [3H]dofetilide Kd was 5 nM) and 200 nM at the sigma receptor (rat striatal tissue). The dissociation of the compound of Example 2(a) from the cloned dopamine D2 receptor was 50% completed by 23 sec, far longer than the 30 minutes for either 2 nM haloperidol or 3 nM chlorpromazine. The in vivo occupancy of dopamine D2 receptors by 10 mg/kg (subcutaneous injection in 0.5 ml of 30% dimethylformamide and 2% glacial acetic acid) of the compound of Example 2(a) was 61% after one hour (Sprague Dawley rats of 300 g). The therapeutic occupancies of D2 in humans was between 60% and 80%. An oral dose of 20 mg/kg of the compound of Example 2(a) resulted in the following D2 receptor occupancy (using a control binding potential of 9.23) as a function of time:

30 min. 39% D2 occupancy
1 hour 63% D2 occupancy
2 hour 24% D2 occupancy
3 hour 28% D2 occupancy
The compound of Example 2(a) did not result in any catalepsy up to 60 mg/kg in mice. This value provides a large therapeutic margin above the therapeutic dose of 3-10 mg/kg.
The compound of Example 2(b) had a dissociation constant, K, of 660 nM at the D2 receptor. Any compound with a K higher than 200 nM at the D2 receptor is of no clinical value in alleviating psychoses.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A compound selected from a compound of Formula (I): 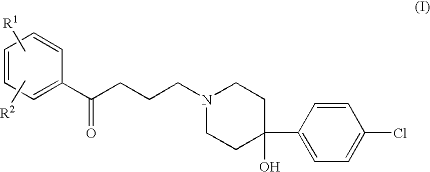
wherein
R¹ is selected from the group consisting of OC₁₋₆alkyl, halo-substituted OC₁₋₆alkyl and OH; and
R² is selected from the group consisting of H and fluoro;
and pharmaceutically acceptable acid addition salts and solvates thereof,
with the proviso that when R¹ is OCH₃, R¹ is attached at the 3-position of the phenyl ring.

2. The compound according to claim 1, wherein R¹ is selected from the group consisting of OC₁₋₄alkyl, fluoro-substituted OC₁₋₄alkyl and OH.

3. The compound according to claim 2, wherein R¹ is selected from the group consisting of OCH₃, OCF₃ and OH.

4. The compound according to claim 3, wherein R¹ is OCH₃.

5. The compound according to claim 1, wherein R² is H.

6. The compound according to claim 1, wherein R² is F attached at the 4-position of the phenyl ring.

7. A compound selected from a compound of Formula I: 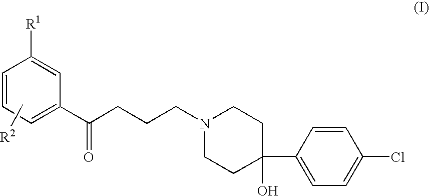
wherein
R¹ is selected from the group consisting of OC₁₋₆alkyl, fluoro-substituted OC₁₋₆alkyl and OH; and
R² is selected from the group consisting of H and fluoro;
and pharmaceutically acceptable acid addition salts and solvates thereof.

8. The compound according to claim 7, wherein R¹ is selected from the group consisting of OC₁₋₄alkyl, fluoro-substituted OC₁₋₄alkyl and OH.

9. The compound according to claim 8, wherein R¹ is selected from the group consisting of OCH₃, OCF₃ and OH.

10. The compound according to claim 9, wherein R¹ is OCH₃.

11. The compound according to claim 7, wherein R² is H.

12. The compound according to claim 7, wherein R² is F attached at the 4-position of the phenyl ring.

13. A compound selected from a compound of Formula I: 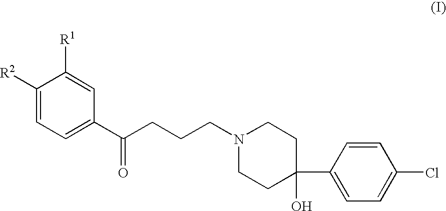
wherein
R¹ is selected from the group consisting of OC₁₋₆alkyl, fluoro-substituted OC₁₋₆alkyl and OH; and
R² is selected from the group consisting of H and fluoro;
and pharmaceutically acceptable acid addition salts and solvates thereof.

14. The compound according to claim 13, wherein R¹ is selected from the group consisting of OC₁₋₄alkyl, fluoro-substituted OC₁₋₄alkyl and OH.

15. The compound according to claim 14, wherein R¹ is selected from the group consisting of OCH₃, OCF₃ and OH.

16. The compound according to claim 15, wherein R¹ is OCH₃.

17. The compound according to claim 13, wherein R² is H.

18. The compound according to claim 13, wherein R² is F.

19. The compound according to claim 1, which is selected from the group consisting of:
4-[4-(4-chlorophenyl)-4-hydroxypiperidin-1-yl]-1-(4-fluoro-3-methoxyphenyl)butan-1-one;
4-[4-(4-chlorophenyl)-4-hydroxypiperidin-1-yl]-1-(3-methoxyphenyl)butan-1-one;
4-[4-(4-chlorophenyl)-4-hydroxypiperidin-1-yl]-1-(3-trifluoromethoxyphenyl)butan-1-one;
4-[4-(4-chlorophenyl)-4-hydroxypiperidin-1-yl]-1-(3-ethoxyphenyl)butan-1-one;
4-[4-(4-chlorophenyl)-4-hydroxypiperidin-1-yl]-1-(4-fluoro-3-ethoxyphenyl)butan-1-one; and
4-[4-(4-chlorophenyl)-4-hydroxypiperidin-1-yl]-1-(4-fluoro-3-trifluoromethoxyphenyl)butan-1-one.

20. The compound according to claim 1, which is selected from the group consisting of:
4-[4-(4-chlorophenyl)-4-hydroxypiperidin-1-yl]-1-(3-methoxyphenyl)butan-1-one;
4-[4-(4-chlorophenyl)-4-hydroxypiperidin-1-yl]-1-(3-trifluoromethoxyphenyl)butan-1-one; and
4-[4-(4-chlorophenyl)-4-hydroxypiperidin-1-yl]-1-(3-ethoxyphenyl)butan-1-one.

21. The compound according to claim 1, wherein the acid addition salt is a hydrochloride salt.

22. A pharmaceutical composition comprising a compound according to claim 1 a pharmaceutically acceptable carrier and/or diluent.

23. A method of treating psychosis comprising administering an effective amount of a compound according to claim 1 to a subject in need thereof.

24. The method according to claim 23, wherein the psychosis is L-DOPA-induced psychosis.
